(19) United States
(12) Patent Application Publication
(10) Pub. No.: US 2009/0182004 A1
(43) Pub. Date: Jul. 16, 2009

(54) Imiquimod formulation

(76) Inventors: Gareth Winckle, Petaluma, CA (US); David W. Osborne, Fort Collins, CO (US)

Correspondence Address: HOWARD EISENBERG, ESQ., 1220 LIMBERLOST LANE, GLADWYNE, PA, 19035, US

(21) Appl. No.: 12/319,978

(22) Filed: Jan. 14, 2009

Related U.S. Application Data
(60) Provisional application No. 61/011,106, filed on Jan. 15, 2008.

(51) Int. Cl. A61K 31/437

(52) U.S. Cl. 514/293

(57) ABSTRACT

Solutions of members of the imidazoquinoline family of drugs, such as imiquimod or an analog thereof, are made by combining the drug in a solvent system containing one or more non-aqueous solvents and a hydrogen bond forming compound, wherein the solvent system contains a low level of water.

This application claims the benefit of pending U.S. Provisional Patent Application Ser. No. 61/011,106, filed Jan. 15, 2008.

### FIELD OF THE INVENTION

The invention pertains to the field of solubility of pharmaceutically active compounds and particularly to the field of enhancing solubility, stability, and skin penetration of imiquimod and other members of the imidazoquinoline family of drugs.

### BACKGROUND OF THE INVENTION

Imiquimod(1-(2-methylpropyl)-1H-imidazo[4,5-c]quinolin-4-amine) is a member of the imidazoquinoline family of drugs. Other members of this family include analogs of imiquimod such as R-848 (resiquimod), R-842 (a hydroxylated metabolite of imiquimod), S-27609, and S-28463. This family of drugs bears a resemblance to nucleoside analogs and has been shown to have the property of immune response modifiers and stimulators, although the exact mechanism of their action is not known.

Imiquimod and its analogs have been shown to be useful when applied topically in the treatment of various skin diseases, including basal cell carcinoma, actinic keratosis, and Bowen's disease (squamous cell carcinoma in situ). Navi and Huntley, Dermatology Online Journal, 10(1):4 (2004). Gerster, U.S. Pat. No. 4,689,338 discloses additional analogs of imiquimod and that imiquimod and its analogs have antiviral efficacy. The efficacy of imiquimod and its analogs as immune response modifiers and for treatment of various skin conditions including tumors and viral diseases are disclosed in Skwierczynski, U.S. Pat. No. 6,245,776, and Wick, U.S. Pat. No. 5,238,944.

Imiquimod is currently marketed for topical application as cream formulation marketed under the name Aldara® Cream (Graceway Pharmaceuticals, Bristol, Tenn.). Aldara® Cream formulation has been approved by the FDA for the treatment of actinic keratosis, basal cell carcinoma, and external genital and perianal warts. Although not approved for this use, Aldara® Cream has also been used to treat cutaneous warts other than genital and perianal warts.

Imiquimod has the structural formula shown below as Formula I.

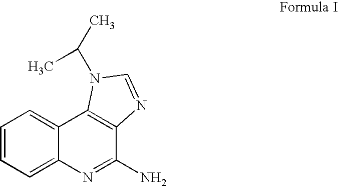

Imiquimod is a planar aromatic molecule which tends to interact with adjacent imiquimod molecules to potentially form stacked arrangements. The strong imiquimod-imiquimod intermolecular forces and stacking tendency makes imiquimod quite insoluble in water and in organic solvents and also renders solutions of imiquimod unstable as adjacent imiquimod molecules in solution interact, stack together, and precipitate out of solution.

Wick, U.S. Pat. No. 5,238,944, and Skwierczynski, U.S. Pat. No. 6,245,776, disclose that oil-in-water emulsion formulations containing imiquimod may be obtained by preparing the oil phase by combining imiquimod with a fatty acid such as isostearic acid or oleic acid. Wick discloses imiquimod pharmaceutical formulations in the form of a cream, an ointment, or a pressure-sensitive adhesive composition. The ointment and pressure-sensitive composition are free of water. Water is present in the cream formulation at a concentration between 45% to 85%.

Skwierczynski discloses formulations containing imiquimod, a fatty acid, an emulsifier, a viscosity enhancing agent, and a preservative. The remainder of the formulation is composed of water.

The presently marketed imiquimod formulation, Aldara® Cream, is an emulsion based upon the disclosure of Wick which contains 5% imiquimod and 25% isostearic acid. The oil-in-water Aldara® Cream emulsion containing imiquimod and isostearic acid presents several problems that need to be addressed. The solution of imiquimod in the oil phase of the emulsion is not stable and, over time, the imiquimod tends to precipitate. It is believed that the lack of physical stability of the imiquimod emulsion is one reason that Aldara® Cream is marketed in single-use packets and that it is recommended that packets that are unused during the treatment period should be discarded.

Secondly, the imiquimod in Aldara® Cream penetrates poorly through non-keratinized human skin and even less readily penetrate through keratinized human skin. Cutaneous warts occurring at locations other than in the genital and perianal areas are more highly keratinized than are genital and perianal warts. Due to the very poor penetrability of imiquimod from the Aldara® Cream formulation through keratinized human skin, treatment of cutaneous warts with Aldara® Cream is often accomplished utilizing an occlusive wrap which, in addition to being clinically cumbersome, has not been proven to significantly enhance efficacy.

Aldara® Cream also is associated with a very high incidence of irritation at the site of administration. A portion of the irritation potential of Aldara®° Cream appears to be due to the presence of high concentration of a fatty acid solvent, such as isostearic acid, that is required to solubilize imiquimod in the oil phase of the emulsion. A second cause of irritation is that due to imiquimod itself. Imiquimod in Aldara® Cream is present at a 5% concentration. Such a high concentration is necessitated due to the poor penetrability of imiquimod through human skin.

Yosha, U.S. Patent Application Publication No. 2007/0264317, addresses the problems associated with the Aldara® Cream formulation and particularly with the poor penetrability of the imiquimod contained therein and the high concentration of isostearic acid. The composition of Yosha contains imiquimod in a micronized form, which is disclosed to be required in order to achieve good penetration of imiquimod. Each of the compositions disclosed in Yosha contains at least 40% water. The composition of Yosha further contains a fatty acid such as oleic acid or linoleic acid in combination with either or both of stearic acid and oleyl alcohol. Yosha does not provide data concerning the irritation potential of the imiquimod formulations containing the oleic acid or linoleic acid. However, liquid fatty acids such as oleic acid are known to be irritating to skin.

A significant need remains for a pharmaceutical formulation containing imiquimod, or an analog thereof, that is physically stable, has reduced irritation potential than prior art formulations containing imiquimod, and that provides improved penetration of imiquimod into skin, and particularly into keratinized skin.

### DESCRIPTION OF THE INVENTION

It has been discovered that the solubility of imiquimod, and its analogs such as R-848, R-842, S-27609, and S-28463, is increased by combining the imiquimod or analog with a suitable hydrogen bond former compound in a solvent system that contains a low level of water and that, preferably, is essentially free of water. The formulation obtained thereby preferably has a low level or is substantially free of fatty acids that are liquid at room temperature. It has been further discovered that preferred formulations of the invention provide enhanced penetration of imiquimod or analog thereof into human skin compared with prior art formulations containing imiquimod.

The present invention therefore provides several solutions to problems associated with prior art formulations containing imiquimod. The present invention provides solutions of imiquimod that have a low level or are substantially free of isostearic acid, and preferably have a low level or are substantially free of any fatty acids that are liquid at room temperature. Thus, irritation due to the presence of such fatty acids, particularly high levels thereof, is no longer a concern when utilizing the formulation of the invention.

Moreover, because preferred formulations of the invention provide an enhanced penetrability of imiquimod into human skin, a lower concentration of imiquimod may be efficaciously utilized than the 5% imiquimod formulation of the prior art that is presently available. Therefore, the irritation potential of imiquimod formulations due to the presence of high concentrations of imiquimod may be significantly reduced when such formulations of the invention are administered. Further, the cost of making an effective pharmaceutical composition of imiquimod or an analog, which compounds are known in the art to be very expensive to synthesize, is substantially reduced.

In this specification, the invention is described primarily in terms of imiquimod. It is to be understood, however, that imiquimod is illustrative of the imidazoquinoline family of drugs and that analogs of imiquimod, including those disclosed herein, are included within the scope of the invention.

The imiquimod, or analog thereof, may be of any particle size prior to incorporation into the formulation of the invention. For example, the imiquimod or analog may be uncontrolled with respect to particle size, or may be coarse, micronized, or nanoparticulate.

The concentration of the constituents of the formulation of the invention is in percent by weight (% w/w). The concentrations of constituents of the formulation of the invention are determined at standard conditions of room temperature and atmospheric pressure at sea level.

The term “low level of water” means an amount of water that is less than that which will prevent the formation of a stable complex between the hydrogen bond former compound and the imiquimod, or analog thereof, in the formulation. Water readily forms extremely stable hydrogen bonds and it is proposed that water would form stable hydrogen bonds with the hydrogen bond former. Consequently, the presence of water, in sufficient quantity, is theorized to successfully and competitively inhibit the formation of hydrogen bonds between imiquimod and the hydrogen bond former compound. Therefore, the purpose of controlling the water content in the formulation of the invention is to reduce or eliminate the competitive hydrogen bond formation between the hydrogen bond former and water and thus to enable the formation of hydrogen bonds between the hydrogen bond former and imiquimod. For purposes of this application, the amount of water that is less than that which will prevent the formation of a stable complex between the hydrogen bond former compound and the imiquimod, or analog thereof, in the formulation is 30% or less of the weight of the formulation. Thus, the formulation of the invention that contains water in an amount that is less than that which will prevent the formation of a stable complex between the hydrogen bond former compound and the imiquimod, or analog thereof may contain 30%, 25%, 20%, 15%, 10%, 5%, or 0% water, or any concentration in between 0% and 30%.

The term “essentially free of water” means that the formulation contains an amount of water that is less than 10% w/w. Preferably, the formulation that is essentially free of water has a concentration of water less than 5% w/w. Even more preferably, the concentration of water is less than 3%. In a particularly preferred embodiment, the concentration of water is about 2% or less.

Preferably, the concentration of water in the formulation of the invention is less than 10 times the dissolved concentration of imiquimod in the formulation. More preferably, the concentration of water is less than 5 times the dissolved concentration of imiquimod. Even more preferably, the concentration of water is less than twice the dissolved concentration of imiquimod. Most preferably, the concentration of water is less than the dissolved concentration of imiquimod. In a particularly preferred embodiment, the concentration of water is less than 50% of the dissolved concentration of imiquimod in the formulation.

Thus, for a formulation containing 1% dissolved imiquimod, it is preferred that the concentration of water should be less than 10%, more preferred less than 5%, even more preferred less than 2%, and most preferred less than 1%. It is particularly preferred that the concentration of water is less than 0.5%.

When utilizing solvents that are azeotropes of water in which the concentration of water in the formulation cannot be reduced to levels described above, it is preferred, although not necessarily essential, that the concentration of water in the solvent system is no more than three times the minimum concentration of water that can be obtained in each individual solvent by distillation. It is more preferable that the concentration of water in the solvent is no more than twice the minimum concentration of water that can be obtained in the individual solvents by distillation. It is most preferred that the concentration of water should be no more than the minimum concentration of water that can be obtained in the individual solvents by distillation. Examples of azeotropes of water include ethanol, glycerin, benzyl alcohol, 1 -N-methyl-2-pyrrolidone (NMP), and propylene glycol.

The term “low level” when referring to isostearic acid or to fatty acids that are liquid at room temperature means that the formulation contains 12.5% or less of such isostearic acid or fatty acid.

The term “substantially free”, when referring to isostearic acid or to fatty acids that are liquid at room temperature, means that the formulation contains 2.5% or less of such isostearic acid or fatty acid. Preferably, the formulation contains 1.0% or less of isostearic acid or fatty acid. More preferably, the formulation contains 0.5% or less. Most preferably, the formulation contains 0.25% or less. And, in a most preferred embodiment, the formulation of the invention is completely free of isostearic acid or other fatty acids that are liquid at room temperature.

The formulation of the invention may contain, if desired, fatty acids such as stearic acid that are solid at room temperature. Such solid fatty acids are mild and are non-irritating to skin. The concentration of such solid fatty acids is not included when determining whether a formulation is substantially free of fatty acids that are liquid at room temperature.

The hydrogen bond former compound of the current invention is a chemical compound that contains at least two sites that are able to form a hydrogen bond with imiquimod or that can donate, or partially donate, a proton to imiquimod in order to provide a non-covalent intermolecular bond with imiquimod, or that can accept, or partially accept, a proton from imiquimod in order to provide a non-covalent intermolecular bond with imiquimod. In this specification, the term “hydrogen bond former compound” is used to mean the hydrogen bond former compound of the invention. It is believed that the hydrogen bond former compound, in combination with imiquimod in a non-aqueous solvent, produces a complex with imiquimod. The complex is more soluble in the non-aqueous solvent than is imiquimod in the absence of the hydrogen bond former compound.

The concentration of the hydrogen bond former compound in the formulation is that which is sufficient to increase the solubility of imiquimod or analog thereof in a formulation that contains a low level of water and that, preferably, is essentially free of water.

The molar ratio of the hydrogen bond former compound and of the imiquimod in the complex may vary depending on the particular hydrogen bond former compound that is utilized and the relative concentrations of imiquimod, hydrogen bond former compound, and water that are present in the solution. It is theorized that a molar ratio in the complex of hydrogen bond former compound and imiquimod of 1:1 is preferred. However, the ratio may be higher than 1:1, for example 2:1, 3:1, or even 4:1. Alternatively, the ratio may be lower than 1:1, for example 1:2, 1:3, or even 1:4. It is further conceived that the molar ratio of hydrogen bond former compound and imiquimod in the complex may be higher than 4:1 or lower than 1:4.

Examples of suitable hydrogen bond former compounds for the method and formulations of the present invention include, but are not limited to, alpha-hydroxy acids such as lactic acid and glycolic acid; beta-hydroxy acids such as salicylic acid and gentisic acid; alkyl-sarcosinates such as cocoyl sarcosine and N-laroyl sarcosine; anionic pegylated dimethicone derivatives such as dimethicone PEG-7 phthalate, dimethicone PEG-7 succinate, and dimethicone PEG-8 phosphate; anionic oleyl ether surfactants such as oeth-3 phosphate; anionic laureth ether surfactants such as laureth-4 carboxylic acid; cyclic acids such as benzoic acid and gallic acid; and cyclic acidic sugars such as glucuronic acid.

In accordance with the method of the invention for making a solution containing imiquimod, imiquimod and one or more hydrogen bond former compounds are combined in a non-aqueous solvent system that contains a low level of water, and that preferably is essentially free of water. The saturated dissolved concentration of the imiquimod obtained thereby is higher than the saturated dissolved concentration of imiquimod in an identical non-aqueous solvent system in which the one or more hydrogen bond former compounds are not combined.

The non-aqueous solvent system of the invention is any solvent system in which the interaction of imiquimod and the hydrogen bond former compound may occur. Thus, it is believed that practically any solvent system that contains a low level of water, such as an essentially non-aqueous solvent system, may be utilized in accordance with the invention. It is theorized that solvent systems containing one or more polar solvents may provide better solubility of imiquimod by interacting with the imiquimod or with the hydrogen bond former compound. It is also theorized that the polar anhydrous solvent system of the invention may further contribute to the inhibition of imiquimod-imiquimod interactions. Additionally, polar solvents may be more capable of dissolving the hydrogen bond former compound and the complex containing the imiquimod and the hydrogen bond former compound. Therefore, polar solvents are preferred over non-polar solvents. The solvent system of the invention may include only a single solvent. Alternatively, the solvent system of the invention may include a multiplicity of solvents.

The solvent system of the invention should be pharmaceutically acceptable and should possess some degree of inherent solubility for imiquimod that is higher than the inherent solubility of imiquimod in water and should also possess some degree of inherent solubility for the hydrogen bond former compound or compounds. Thus, the solvent system facilitates interaction between the imiquimod and the hydrogen bond former compounds.

Examples of suitable solvents for the solvent system of the invention include, but are not limited to, aprotic solvents such as NMP and dimethyl sulfoxide (DMSO); cyclic alcohols such as benzyl alcohol; short chain liquid alcohols such as ethanol and diols or triols such as propylene glycol, glycerin, and butylene glycol; esters such as myristyl lactate, isopropyl myristate, and ethyl acetate; ethers such as diethylene glycol monoethyl ether (i.e. Transcutol®, Gattefosse, Gennevilliers, France) and dimethyl isosorbide; pharmaceutical oils such as triglycerides; and silicones such as volatile or non-volatile silicones such as dimethicone and cyclomethicone, respectively.

The solution of the invention is physically stable. Dissolved levels of imiquimod are determined at steady state level 12 weeks after making the solution. Further, the compositions of the invention have been found to be essentially free of precipitate of imiquimod after 12 weeks of aging at 25° C., 40° C., or 50° C.

The combination of preferred hydrogen bond former compounds and preferred non-aqueous solvents listed above resulted in unexpected and significantly enhanced topical delivery and skin penetration relative to the prior art product (Aldara® Cream). Statistically significantly greater delivery was achieved while utilizing a substantially lower imiquimod loading dose in compositions embodying the invention. It is proposed that these results were achieved due to a combination of enhanced imiquimod solubility in the skin and solubilization/fluidization of the stratum comeum lipids mediated by the solvents and hydrogen bond former compounds of the invention.

In addition to the imiquimod and the hydrogen bond former compound, the solution of the invention may contain a polymer. The polymer may act as a thickening agent and may enhance the stability of the imiquimod solution of the invention. It is theorized that polymeric agents, for example hydroxypropyl cellulose (HPC), carbomers (carboxy vinyl polymers), and polyvinyl pyrrolidone, may form hydrogen bond type interactions with ‘free’ imiquimod, thereby serving as solubilizers and anti-nucleating agents. Additionally, polymers may present a steric hindrance to the interaction of adjacent imiquimod molecules. These interactions are thought to enhance the physical stability of imiquimod in the preferred solvent systems of the invention and, in some cases such as with HPC, also providing enhanced viscosity.

Preferably, the polymer should have a solubility of at least 0.01% in the solvent system of the formulation. More preferably, the polymer has a solubility of at least 0.05% in the solvent system. Most preferably, the polymer has a solubility of at least 0. 10% in the solvent system. If a polymer is included in the formulation, it is preferred, but not essential, that the polymer have the potential to combine with imiquimod in a non-covalent bond, such as a hydrogen bond. Such interaction will further act to stabilize the solution of the invention.

Examples of polymers that are suitable for the solution of the invention include cellulose derivatives, such as hydroxypropyl cellulose, ethylcellulose, hydroxypropyl methyl cellulose and hydroxypropyl ethylcellulose; methacrylic acid copolymers such as those marketed under the tradename Eudragit® (Evonik Industries AG, Essen, Germany); carbomers such as those marketed under the tradenames Carbopol® or Pemulen® (Lubrizol Advanced Materials, Inc., Cleveland, Ohio); pyrrolidone-containing polymers such as polyvinyl pyrrolidone (PVP); polyoxyethylene such as polyethylene glycol, such as PEG 400, and Polyox™ (The Dow Chemical Co., Midland, Mich.); polyoxyethylene/polyoxypropylene block co-polymers such as poloxamers (BASF Corporation, Florham Park, N.J. USA), and polyvinyl alcohols. The concentration of the polymer is preferably less than 10% w/w of the formulation. More preferably, the concentration is less than 5% and most preferably less than 2.5%. In a most preferred embodiment, the concentration of the polymer is 1% or less.

The formulations of the invention may further include pharmaceutically acceptable polymeric and/or non-polymeric excipients typically used in formulations and known to those skilled in the art. Such excipients include, for example, thickening and/or gelling agents, fatty ester based or waxy gelling agents, humectants, emollients, pH stabilizing agents, preservatives, and anti-oxidants.

The formulation of the invention is preferably a solution. If desired, however, the solution of the invention may form a portion of the formulation of the invention. For example, the solution may constitute an internal or an external phase of an emulsion, particularly of a non-aqueous emulsion.

With preferred formulations of the invention, it has been unexpectedly determined that imiquimod from these formulations has enhanced permeation when applied topically to skin. Such formulations contain imiquimod, one or more hydrogen bond former compounds as described above, a solvent system as described above, plus optional excipients as described above. It is conceived that virtually any formulation of the invention will provide increased skin penetration of imiquimod compared to the Aldara® formulation of the prior art. In the Examples that follow, 9 different formulations of the invention were made and tested for skin penetration of imiquimod. Eight of the formulations tested provided enhanced skin penetration of imiquimod compared to the penetration of imiquimod from Aldara® Cream.

In the Examples the Aldara® Cream formulation tested contained 5% imiquimod whereas each of the formulations of the invention contained only 1% imiquimod. In eight of nine formulations tested, the % dose applied of imiquimod that penetrated was found to be higher than that from Aldara® Cream. In five of these eight, the % dose applied of imiquimod that penetrated was found to be at least 5 times higher than that from Aldara® Cream. Therefore, even though the test formulations contained only 20% as high a concentration of imiquimod as Aldara® Cream, the absolute amount of imiquimod that was found to penetrate skin was higher than that from Aldara® Cream.

Accordingly, in another embodiment, the invention is a method for providing increased skin penetration of imiquimod. According to this embodiment, a formulation comprising the solution of the invention is obtained and is topically applied to skin.

The invention is further illustrated in the following non-limiting examples.

### EXAMPLE 1

### Imiquimod Saturated Solubility in Individual Liquid Excipients

Solutions of imiquimod were prepared using various individual excipients as shown below in Table 1. The solutions were prepared with excess imiquimod and incubated for approximately 1 week at 25° C., under constant agitation. Excess imiquimod was removed by centrifugation or filtration and the concentration of Imiquimod in the clear supernatant was determined by HPLC-UV. As shown in Table 1, imiquimod exhibits a wide range of solubility in different classes of neat liquid excipients.

TABLE 1

| | Imiquimod Saturated Solubility |
|---|---|
| Component | (% w/w, 25° C.) |
| Lactic acid | 18.22 |
| Oleic acid | 17.58 |
| Cocoyl sarcosine | 11.45 |
| Dimethicone PEG-7 Phthalate | 7.71 |
| Oleth 3 phosphate (O-3 P) | 5.66 |
| Dimethicone PEG-7 Succinate | 2.78 |
| Laureth 4 carboxylic acid | 1.01 |
| Benzyl alcohol | 0.44 |
| 1-N-Methyl-2-Pyrrolidone (NMP) | 0.17 |
| Trilaureth-4 Phosphate | 0.14 |
| Transcutol | 0.13 |
| Dimethicone PEG-8 Phosphate | 0.13 |
| Glycerin | 0.12 |
| Myristyl lactate | 0.09 |
| Dimethyl sulfoxide (DMSO) | 0.09 |
| Propylene glycol | 0.06 |
| Ethanol (200 proof) | 0.03 |
| Brij 93 (polyoxyethylene 2 oleyl ether) | 0.02 |
| Isopropyl alcohol | 0.02 |
| Poloxamer 124 | 0.02 |
| Cyclomethicone | 0.004 |
| Water | <0.0001 |

### EXAMPLE 2

### Enhanced Solubility Mediated by Various Hydrogen Bond Forming Compounds in Various Solvents

The solubilizing effects of various hydrogen bond forming compounds in simple systems were evaluated using N-Methyl Pyrrolidone (NMP) or Dimethyl Sulfoxide (DMSO) as solvents. Hydrogen bond forming compound/solvent solutions were prepared with excess imiquimod and incubated for up to 12 weeks at 25° C., 40° C. and 50° C. Samples were removed at initial, 4 and 12 week intervals. Excess imiquimod was removed by centrifugation or filtration and the concentration of imiquimod in the clear supernatant was determined by HPLC-UV.

### EXAMPLE 2A

### NMP

Various formulations containing 5% imiquimod, the solvent NMP and a hydrogen bond forming compound were made. The components of these formulations are shown in Table 2. The dissolved concentration of imiquimod in each formulation at 25° C following incubation at 40° C. for 12 weeks is shown in Table 3.

TABLE 2

| | Formulation ID 1993- | | | | | | |
|---|---|---|---|---|---|---|---|
| | 110A | 110D | 110C | 111A | 111B | 111C | 120A |
| Components | % w/w | | | | | | |
| Lactic Acid | 5.00 | | | | | | |
| Glycolic Acid (Glypure 99) | | 4.20 | | | | | |
| Salicylic Acid | | | 7.70 | | | | |
| Gentisic Acid | | | | 8.60 | | | |
| Gallic Acid | | | | | 10.60 | | |
| Glucuronic Acid | | | | | | 10.80 | |
| Glycolic Acid Ethoxylate Lauryl Ether | | | | | | | 19.05 |
| 1-N-Methyl-2-Pyrrolidone (NMP) | 90.00 | 90.80 | 87.30 | 86.40 | 84.40 | 84.20 | 75.95 |
| Imiquimod | 5.00 | 5.00 | 5.00 | 5.00 | 5.00 | 5.00 | 5.00 |
| Total | 100.00 | 100.00 | 100.00 | 100.00 | 100.00 | 100.00 | 100.00 |

TABLE 3

| | | Dissolved Imiquimod | | |
|---|---|---|---|---|
| | | (% w/w) | | |
| Formulation ID | Formulation Description | Initial | 4 weeks | 12 weeks |
| 1993-110A | NMP/Lactic Acid | 2.16 | 1.71 | 1.85 |
| 1993-110D | NMP/Glycolic acid | 1.44 | 0.99 | 1.81 |
| 1993-110C | NMP/Salicylic Acid | 4.18 | 3.06 | 2.92 |
| 1993-111A | NMP/Gentisic Acid | 3.33 | 2.15 | 2.22 |
| 1993-111B | NMP/Gallic Acid | 0.68 | 0.69 | 0.84 |
| 1993-111C | NMP/Glucuronic Acid | 2.94 | 1.46 | 2.13 |
| 1993-120A | NMP/Glycolic Acid | 1.50 | 1.26 | 1.39 |
| | Ethoxylate Lauryl Ether | | | |

As shown in Table 3, the imiquimod concentration in the supernatant of NMP/hydrogen bond forming compound solutions decreased between the initial and 4 week sampling points. However, the imiquimod concentration then appeared to plateau between the 4 and 12 week sampling intervals. The extent of imiquimod solubility enhancement afforded by the incorporation of a hydrogen bond forming compound in NMP is summarized in Table 4, which shows the increase in imiquimod solubility of imiquimod in formulations containing NMP and a hydrogen bond former compound relative to the imiquimod solubility in NMP alone, following storage at 25° C. for 12 weeks. The data was calculated relative to the saturated solubility of imiquimod in 100% NMP (0.17% w/w).

TABLE 4

| | | T = 0 | T = 12 weeks |
|---|---|---|---|
| | | Imiquimod Solubility | |
| | | increase relative to 100% | |
| Formulation ID | Formulation description | NMP | |
| 1993-110A | NMP/Lactic Acid | 11.8 | 8.7 |
| 1993-110C | NMP/Salicylic Acid | 23.8 | 16.4 |
| 1993-110D | NMP/Glycolic acid | 7.6 | 5.1 |
| 1993-111A | NMP/Gentisic Acid | 18.8 | 16.5 |
| 1993-111B | NMP/Gallic Acid | 3.0 | 2.4 |
| 1993-111C | NMP/Glucuronic Acid | 16.4 | 13.7 |
| 1993-120A | NMP/Laureth-3 Glycolic Acid | 7.9 | 6.1 |

### EXAMPLE 2B

### DMSO

Various formulations containing 5% imiquimod, the solvent DMSO and a hydrogen bond forming compound were made. The components of these formulations are shown in Table 5. The dissolved concentration of imiquimod in each formulation, determined as previously described, following incubation at 40° C. for 12 weeks is shown in Table 6.

TABLE 5

| | Formulation ID 2494- | | | | | | | | |
|---|---|---|---|---|---|---|---|---|---|
| | 32A | 32B | 32C | 33A | 33B | 33C | 34A | 35A | 35B |
| Components | % w/w | | | | | | | | |
| Lactic Acid | 5.0 | | | | | | | | |
| Glycolic Acid | | 4.2 | | | | | | | |
| Salicylic Acid | | | 7.7 | | | | | | |
| Gentisic Acid | | | | 8.6 | | | | | |
| Gallic Acid | | | | | 10.6 | | | | |
| Glucuronic Acid | | | | | | 10.8 | | | |
| Benzoic acid | | | | | | | 6.8 | | |
| Oleth-3 Phosphate | | | | | | | | 5.6 | |
| Cocoyl sarcosine | | | | | | | | | 2.2 |
| Dimethyl Sulfoxide (DMSO) | 90.0 | 90.8 | 87.3 | 86.4 | 84.4 | 84.2 | 88.2 | 92.4 | 92.8 |
| Imiquimod | 5.0 | 5.0 | 5.0 | 5.0 | 5.0 | 5.0 | 5.0 | 2.0 | 5.0 |
| Total | 100.00 | 100.00 | 100.00 | 100.00 | 100.00 | 100.00 | 100.00 | 100.00 | 100.00 |

TABLE 6

| | | Dissolved Imiquimod | | |
|---|---|---|---|---|
| | | (% w/w) | | |
| Formulation ID | Formulation description | Initial | 4 weeks | 12 weeks |
| 2494-32A | DMSO/Lactic Acid | 0.69 | 0.76 | 0.46 |
| 2494-32B | DMSO/Glycolic Acid | 0.66 | 0.67 | 0.51 |
| 2494-32C | DMSO/Salicylic Acid | 6.45 | N/T | 4.67 |
| 2494-33A | DMSO/Gentisic Acid | 6.55 | N/T | 6.89 |
| 2494-33B | DMSO/Gallic Acid | 0.78 | 0.45 | 0.33 |
| 2494-33C | DMSO/Glucuronic Acid | 1.99 | 1.52 | 0.99 |
| 2494-34A | DMSO/Benzoic Acid | 0.68 | 0.73 | 0.44 |
| 2494-35A | DMSO/Oleth-3 Phosphate | 2.41 | 1.60 | 1.01 |
| 2494-35B | DMSO/Cocoyl Sarcosine | 0.42 | 0.33 | 0.04 |

N/T—Not tested

As shown in Table 6, the combinations of DMSO/salicylic acid and DMSO/gentisic acid exhibited markedly greater imiquimod solubility than did the other DMSO/hydrogen bond former compound combinations, and the solubility remained relatively constant over the 12 weeks of the study.

The extent of imiquimod solubility enhancement afforded by the incorporation of a hydrogen bond forming compound in DMSO is summarized in Table 7, which shows the increase in imiquimod solubility of imiquimod in formulations containing DMSO and a hydrogen bond former compound relative to the imiquimod solubility in DMSO alone, following storage at 25° C. for 12 weeks. The data was calculated relative to the saturated solubility of imiquimod in 100% DMSO (0.09% w/w).

TABLE 7

| | | T = 0 | T = 12 weeks |
|---|---|---|---|
| | | Imiquimod Solubility | |
| | | increase relative to 100% | |
| Formulation ID | Formulation description | DMSO | |
| 2494-32A | DMSO/Lactic Acid | 6.6 | 4.0 |
| 2494-32B | DMSO/Glycolic Acid | 6.2 | 4.6 |
| 2494-32C | DMSO/Salicylic Acid | 69.7 | 50.2 |
| 2494-33A | DMSO/Gentisic Acid | 70.8 | 74.6 |
| 2494-33B | DMSO/Gallic Acid | 7.6 | 2.6 |
| 2494-33C | DMSO/Glucuronic Acid | 20.8 | 9.9 |
| 2494-34A | DMSO/Benzoic Acid | 6.5 | 3.8 |
| 2494-35A | DMSO/Oleth-3 Phosphate | 25.4 | 10.1 |
| 2494-35B | DMSO/Cocoyl Sarcosine | 3.6 | −0.5 |

n/a—not available

The initial saturated solubilities of imiquimod in the NMP/hydrogen bond forming compound formulations of Example 2A and in the DMSO/hydrogen bond forming compound formulations of Example 2B were compared and are shown below in Table 8. The saturated solubilities of imiquimod in these formulations following storage for 12 weeks at 25° C. are shown below in Table 9

TABLE 8

| | SOLVENT: NMP | SOLVENT: DMSO |
|---|---|---|
| Hydrogen Bond | Initial Solubility | |
| Forming Compound | Dissolved Imiquimod % w/w | |
| Lactic Acid | 2.16 | 0.69 |
| Glycolic Acid | 1.44 | 0.66 |
| Salicylic Acid | 4.18 | 6.45 |
| Gentisic Acid | 3.33 | 6.55 |
| Gallic Acid | 0.68 | 0.78 |
| Glucuronic Acid | 2.94 | 1.99 |
| Benzoic Acid | n/a | 0.68 |
| Oleth-3 phosphate | n/a | 2.41 |
| Cocoyl Sarcosine | n/a | 0.42 |
| Laureth-3 Glycolic Acid | 1.5 | n/a |

n/a—not available

TABLE 9

| | SOLVENT: NMP | SOLVENT: DMSO |
|---|---|---|
| Hydrogen Bond | Solubility after storage at 25° C., 12 weeks | |
| Forming Compound | Dissolved Imiquimod % w/w | |
| Lactic Acid | 1.63 | 0.40 |
| Glycolic Acid | 1.02 | 0.38 |
| Salicylic Acid | 2.93 | 5.15 |
| Gentisic Acid | 2.94 | n/a |
| Gallic Acid | 0.57 | 0.20 |
| Glucuronic Acid | 2.48 | 0.57 |
| Benzoic Acid | n/a | 0.38 |
| Oleth-3 phosphate | n/a | 1.03 |
| Cocoyl Sarcosine | n/a | 0.04 |
| Laureth-3 Glycolic Acid | 1.2 | n/a |

n/a—not available

The study of this Example establishes that the compositions of the invention, containing the solvents and hydrogen bond forming compounds of the invention, provide for enhanced solubility of imiquimod and that the enhanced solubility of imiquimod is stable, as determined by storage for a period of 12 weeks at 25° C.

### EXAMPLE 3

### Enhancement of Imiquimod Solubility in Solvents with Different Liquid Hydrogen Bond Forming Compounds

Following the solubility study of Example 1, four of the excipients of Table 10 were utilized as liquid hydrogen bond forming compounds in combination with various solvents. The four hydrogen bond forming compounds tested were oleth-3 phosphate (O-3P), cocoyl sarcosine, dimethicone PEG-7 phthalate, and trilaureth-4 phosphate (Table 1). Single solvent systems of varying polarity and solubility were utilized with the four hydrogen bond forming compounds as solubility enhancers. Isopropyl alcohol is a polar, volatile solvent with low solubility for imiquimod. NMP is a polar, non-volatile solvent with somewhat higher solubility for imiquimod. Myristyl lactate is a relatively non-polar and non-volatile solvent with solubilizing capacity for imiquimod similar to that of NMP.

Solutions of a hydrogen bond forming compound and solvent were prepared with excess imiquimod and were incubated for approximately 1 week at 25° C., under constant agitation. Excess imiquimod was removed by centrifugation or filtration and the concentration of imiquimod in the clear supernatant was determined by HPLC-UV. The composition of each solution and the imiquimod solubility are shown in Table 10.

TABLE 10

| | (Formula ID) Batch #: 1993- | | | | | | |
|---|---|---|---|---|---|---|---|
| | 136A | 137A | 137D | 138C | 140A | 140D | 142C |
| Component | % w/w | | | | | | |
| Imiquimod | 2.0 | 2.0 | 2.0 | 2.0 | 2.0 | 2.0 | 2.0 |
| Isopropyl alcohol | 92.4 | | | | | | 86.2 |
| NMP | | 92.4 | 88.0 | | 86.2 | 78.0 | |
| Myristyl lactate | | | | 95.8 | | | |
| Oleth-3 phosphate | 5.6 | 5.6 | | | 11.8 | | 11.8 |
| Trilaureth-4 phosphate | | | | | | | |
| Cocoyl sarcosine | | | | 2.2 | | | |
| Dimethicone PEG-7 phthalate | | | 10.0 | | | 20.0 | |
| Total | 100.0 | 100.0 | 100.0 | 100.0 | 100.0 | 100.0 | 100.0 |
| Observed saturated solubility (% w/w, | 1.39 | 1.98 | 0.61 | 1.90 | 1.88 | 0.98 | 1.73 |
| 25° C.) | | | | | | | |
| Calculated ideal saturated solubility | 0.33 | 0.47 | 0.92 | 0.34 | 0.81 | 1.67 | 0.68 |
| Observed saturated solubility/ | 4.2 | 4.2 | (0.6) | 5.6 | 2.3 | (0.6) | 2.5 |
| calculated ideal saturated solubility | | | | | | | |

As shown in Table 10, the combination of solvent and hydrogen bond forming compound provided a significant increase in imiquimod solubility compared to the calculated ideal solubility based on the sum of the individual solubilities of imiquimod in the solvent and the liquid hydrogen bond forming compound. This data establishes unexpected synergistic solubilization of imiquimod when utilizing a combination of the invention. The data also shows a decrease in saturated solubility relative to calculated ideal solubility when the combination of NMP and dimethicone PEG-7 phthalate was used. This suggests that the combination of NMP and dimethicone PEG-7 phthalate does not produce this synergy in imiquimod dissolution, although the use of dimethicone PEG-7 phthalate as a hydrogen bond forming compound in combination with a different solvent may produce such a synergy.

### EXAMPLE 4

### Enhanced Solubility Mediated by Various Solvent Systems Containing a Hydrogen Bond Former Compound

Several solvent mixtures were prepared to assess the solubilizing capacity for imiquimod of formulations containing polar solvent mixtures essentially free of water and an illustrative hydrogen bond forming compound, oleth-3 phosphate.

Hydrogen bond forming compound/solvent solutions were prepared with excess imiquimod and incubated for approximately 1 week at 25° C., under constant agitation. Excess imiquimod was removed by centrifugation or filtration and the concentration of imiquimod in the clear supernatant was determined by HPLC-UV. The compositions of the formulations containing a blend of solvents and the hydrogen bond forming compound oleth-3 phosphate, and the imiquimod solubility in each formulation, are shown in Table 11.

TABLE 11

| | (Formula ID) Batch # 1993- | | | | |
|---|---|---|---|---|---|
| | 146A | 147A | 148A | 149A | 150A |
| Component | % w/w | | | | |
| Imiquimod | 2 | 3 | 2 | 2 | 4 |
| NMP | 30 | 30 | 30 | 30 | 30 |
| Oleth-3 Phosphate | 11.2 | 11.2 | 11.2 | 11.2 | 11.2 |
| Dimethyl isosorbide | 15 | 15 | 15 | 15 | |
| Isopropyl alcohol | 41.8 | 20 | | | |
| Benzyl alcohol | | 20.8 | 41.8 | 21.8 | 34.8 |
| Ethanol 200 proof | | | | 20 | 20 |
| Total | 100.0 | 100.0 | 100.0 | 100.0 | 100.0 |
| Observed saturated | 1.81 | 2.76 | 1.80 | 1.90 | 3.86 |
| solubility | | | | | |
| (% w/w, 25° C.) | | | | | |
| Calculated ideal | 0.72 | 0.81 | 0.90 | 0.82 | 0.84 |
| saturated solubility | | | | | |
| Observed saturated | 2.5 | 3.4 | 2.0 | 2.3 | 4.6 |
| solubility/calculated | | | | | |
| ideal saturated solubility | | | | | |

As shown in Table 11, the combination of solvent blend and a representative hydrogen bond forming compound, oleth-3 phosphate, provided a significant increase in imiquimod solubility compared to the calculated ideal solubility based on the sum of the individual solubilities of imiquimod in each of the solvents of the blend and in the oleth-3 phosphate. This data establishes the synergy that is obtained in the solubilization of imiquimod when utilizing such a combination containing a blend of anhydrous solvents according to the invention.

### EXAMPLE 5

### Enhanced Stability of Solutions of the Invention Containing Imiquimod

The stability of selected compositions of the invention containing 1% w/w imiquimod was evaluated. The compositions of the formulae assessed are summarized in Table 12.

TABLE 12

| | Formula ID 2494- | | | | | | | | | | |
|---|---|---|---|---|---|---|---|---|---|---|---|
| | 58A | 58C | 59A | 59B | 59C | 72A | 72B | 72C | 73A | 73B | 73C |
| Components | % w/w | | | | | | | | | | |
| Imiquimod | 1 | 1 | 1 | 1 | 1 | 1 | 1 | 1 | 1 | 1 | 1 |
| NMP | 30 | 30 | 30 | 30 | 30 | | 30 | 30 | | 30 | |
| Benzyl alcohol | 25 | 25 | 25 | 25 | 25 | 25 | 25 | 25 | | | |
| Oleth-3 | 5.6 | | | | 5.6 | | | | | | |
| Phosphate | | | | | | | | | | | |
| Cocoyl Sarcosine | | | | 5 | 10 | | 5 | 10 | | | |
| Lactic acid | | 5 | | | | | | | 10 | 10 | 10 |
| Salicylic acid | | | 7.7 | | | | | | | | |
| Ethanol 200 | 38.4 | 39 | 36.3 | 39 | 34 | | | | | | |
| proof | | | | | | | | | | | |
| Transcutol | | | | | | 25 | | | 25 | 25 | |
| Glycerin | | | | | | 43.4 | 39 | 34 | | | |
| Dimethyl | | | | | | | | | | 15 | |
| Isosorbide | | | | | | | | | | | |
| Ethyl acetate | | | | | | | | | | 19 | 30 |
| Myristyl lactate | | | | | | | | | 64 | | 59 |
| | 100.0 | 100.0 | 100.0 | 100.0 | 100.0 | 100.0 | 100.0 | 100.0 | 100.0 | 100.0 | 100.0 |

Each of the formulations was incubated in glass scintillation vials for up to 12 weeks at 5/50° cycling, 25°, 40°, 50°, and 70° and assessed using HPLC-UV for chemical and physical stability over this time period. Results for 50° incubation, which were representative of the data obtained for all storage conditions, are shown in Table 13. The data, as shown in Table 13, establishes that no significant changes in imiquimod concentration were observed following incubation at real time and accelerated conditions. This data indicated that the compositions exhibited favorable physical and chemical stability.

TABLE 13

| Formulation ID | Initial | 4 weeks | 12 weeks |
|---|---|---|---|
| 2494-58A | 0.94 | 1.02 | 0.94 |
| 2494-58C | 0.97 | 1.04 | 0.96 |
| 2494-59A | 0.97 | 0.97 | 0.97 |
| 2494-59B | 1.03 | 1.06 | 1.01 |
| 2494-59C | 1.03 | 1.11 | 1.01 |
| 2494-72A | 0.96 | 1.03 | 0.97 |
| 2494-72B | 0.95 | 1.10 | 0.95 |
| 2494-72C | 0.97 | 1.13 | 0.97 |
| 2494-73A | 0.99 | 1.04 | 0.97 |
| 2494-73B | 1.01 | 1.05 | 1.01 |
| 2494-73C | 1.41 | 1.45 | 1.41 |

### EXAMPLE 6

### Enhanced Imiquimod Saturated Solubility

Several solvent mixtures of the invention were prepared to assess the solubilizing capacity of polar solvent compositions essentially free of water containing preferred hydrogen bond forming compounds. Solutions containing a blend of a multiplicity of non-aqueous solvents and a hydrogen bond forming compound were prepared with excess imiquimod and incubated for approximately 1 week at 25° C., under constant agitation. Excess imiquimod was removed by centrifugation or filtration and the concentration of imiquimod in the clear supernatant was determined by HPLC-UV. The composition of each solution and the solubility of imiquimod in each solution are shown in Table 14.

TABLE 14

| | | Formulation ID 2592- | | | | |
|---|---|---|---|---|---|---|
| | Saturated solubility | 59A | 59B | 59C | 60A | 60B |
| Component | (% w/w, 25° C.) | % w/w | | | | |
| NMP | 0.168 | 30 | 30 | 30 | | |
| Benzyl Alcohol | 0.443 | 25 | 25 | 25 | | |
| Ethanol | 0.029 | 39.4 | 37.3 | | | |
| Glycerin | 0.119 | | | 35 | | |
| Transcutol | 0.131 | | | | 25 | |
| Myristyl lactate | 0.093 | | | | 65 | 60 |
| Ethyl acetate | 0.007 | | | | | 30 |
| Oleth-3 phosphate | 5.655 | 5.6 | | | | |
| Salicylic acid | N/A | | 7.7 | | | |
| Cocoyl sarcosine | 11.449 | | | 10 | | |
| Lactic acid | 18.223 | | | | 10 | 10 |

| | | | | | |
|---|---|---|---|---|---|
| Calculated ideal solubility (% w/w) | 1.072 | 0.754 | 1.930 | 0.250 | 0.695 |
| Saturated Solubility (% w/w, 25° C.) | 2.046 | 1.125 | 4.907 | 1.038 | 0.945 |
| Solubility enhancement ratio* | 1.9 | 1.5 | 2.5 | 4.2 | 1.4 |

*solubility enhancement ratio = saturated solubility/calculated ideal solubility

The data of Table 14 show that an enhancement above calculated ideal solubility was obtained with formulations 59A, 59B, and 59C. This enhancement above ideal solubility was not observed with formulations 60A and 60B. This data shows that, although a particular solvent in combination with a hydrogen bond former compound produces an increase in imiquimod solubility, when utilizing a blend of solvents, such solvent systems must be optimized experimentally.

### EXAMPLE 7

### Enhancement of Skin Penetration of Imiquimod

A study was performed to characterize the in vitro percutaneous penetration of (¹⁴C)-Imiquimod from nine formulations of the invention compared to that from a commercial imiquimod product, Aldara® Cream, following topical application to excised human skin. The compositions of the formulations tested are shown in Table 15. Formulations of the invention contained 1% w/w imiquimod while Aldara® Cream contains 5% w/w imiquimod.

TABLE 15

| | | Formula ID 2592- | | | | | | | | |
|---|---|---|---|---|---|---|---|---|---|---|
| | N/A | 15A | 15B | 15C | 16A | 16B | 16C | 17A | 17B | 17C |
| Component | % w/w | | | | | | | | | |
| Imiquimod | ALDARA | 1.0 | 1.0 | 1.0 | 1.0 | 1.0 | 1.0 | 1.0 | 1.0 | 1.0 |
| N-Methyl pyrrolidone | | | 30.0 | 30.0 | 30.0 | 30.0 | | 30.0 | 30.0 | |
| (NMP) | | | | | | | | | | |
| Dimethyl Sulfoxide | | 91.3 | | | | | | | | |
| (DMSO) | | | | | | | | | | |
| Benzyl alcohol | | | 25.0 | 25.0 | 25.0 | 25.0 | 25.0 | 25.0 | | |
| Oleth-3 Phosphate | | | 5.6 | | | | 5.6 | | | |
| Cocoyl Sarcosine | | | | | | 10.0 | | 10.0 | | |
| Lactic acid | | | | 5.0 | | | | | 10.0 | 10.0 |
| Salicylic acid | | 7.7 | | | 7.7 | | | | | |
| Oleic Acid | | | | | | | | | | |
| Ethanol 200 proof | | | 38.4 | 39.0 | 36.3 | 34.0 | | | | |
| Transcutol | | | | | | | 25.0 | | 25.0 | |
| Glycerin | | | | | | | 43.4 | 34.0 | | |
| Dimethyl Isosorbide | | | | | | | | | 15.0 | |
| Ethyl acetate | | | | | | | | | 19.0 | 30.0 |
| Myristyl lactate | | | | | | | | | | 59.0 |
| | Total | 100.0 | 100.0 | 100.0 | 100.0 | 100.0 | 100.0 | 100.0 | 100.0 | 100.0 |
| % Applied dose | 0.09 | 1.64 | 1.21 | 0.14 | 0.45 | 0.33 | 0.37 | 0.72 | 0.03 | 0.48 |
| penetrated | | | | | | | | | | |
| Mean amount | 215 | 820 | 603 | 67.5 | 227 | 163 | 186 | 361 | 16.1 | 241 |
| penetrated (ng/cm²) | | | | | | | | | | |

Dermatomed human skin was obtained from a single donor following elective abdominoplasty. The tissue was dosed with 5 mg/cm² of formulation spiked with radiolabeled imiquimod at a nominal 1.0 μCi/dose, corresponding to a nominal 3.2 mg dose per cell. Percutaneous absorption was evaluated by mounting the dermatomed tissue in Bronaugh flow-through diffusion cells at 32° C. Five replicates were performed for each formulation. Fresh receptor fluid, PBS containing 0.1% w/v sodium azide and 1.5% w/v oleth-20, was continuously pumped under the skin at a nominal flow rate of 1 ml/hr and collected in 6-hour intervals. Following a 24-hour exposure period, the residual formulation remaining on the skin surface was removed by repeated tape stripping (3 strips/cell). Subsequently, the epidermis was physically separated from the dermis by gentle peeling. The quantity of radioactivity in the tape-strips, epidermis, dermis, and receptor phase samples was determined using liquid scintillation analyzing techniques. Dose recovery (accountability) at the end of the study ranged from 87.6 to 101 percent of the applied dose. Tissue permeation data (% applied dose penetrated and mean amount penetrated, ng/cm²) is presented in the bottom section of Table 15.

As shown in Table 15, receptor phase levels of (¹⁴C)-Imiquimod from Aldara® Cream i.e. material that penetrated the skin, was 0.09±0.04 percent of the applied dose (215±90 ng/cm²). Tissue permeation from the nine formulations of the invention containing 1% imiquimod ranged from 0.03 to 1.64 percent of the applied dose (equivalent to 16.1 ng/cm² and 820 ng/cm² of imiquimod) from Formulations 2592-17B and 2592-15A, respectively. Thus, not only did a higher percentage of the applied dose of imiquimod penetrate into skin from 8 of the 9 formulations tested, but the absolute amount of imiquimod that penetrated into skin from 7 of the 9 formulations of the invention was about equal to or higher that that which penetrated into skin from the Aldara® Cream, even though the amount of imiquimod in the formulations of the invention contained only 20% of that contained in the Aldara® Cream formulation.

### EXAMPLE 8

### Enhanced Solubility of Imiquimod with Multiple Proton Donors

Several solvent mixtures were prepared to assess the solubilizing capacity of polar solvent compositions essentially free of water containing a multiplicity of hydrogen bond forming compounds. Hydrogen bond forming compounds/solvent solutions were prepared with excess imiquimod and incubated for approximately 1 week at 25° C., under constant agitation. Excess imiquimod was removed by centrifugation or filtration and the concentration of imiquimod in the clear supernatant was determined by HPLC-UV. The composition of each solution and the solubility of imiquimod in each solution are shown in Table 16.

TABLE 16

| | | Formulation ID # | | | |
|---|---|---|---|---|---|
| | Neat saturated | 2592-88A | 2592-88B | 2592-89A | 2592-89B |
| Component | solubility (% w/w) | % w/w | | | |
| NMP | 0.1683 | 30 | 30 | 30 | 30 |
| Benzyl Alcohol | 0.4427 | 25 | 25 | 25 | 25 |
| Ethanol | 0.0294 | 31.7 | 29.4 | | |
| Glycerin | 0.1185 | | | 27.3 | 25 |
| Oleth-3 phosphate | 5.6554 | 5.6 | 5.6 | | |
| Salicylic acid | N/A† | 7.7 | | 7.7 | |
| Cocoyl sarcosine | 11.4493 | | | 10 | 10 |
| Lactic acid | 18.2234 | | 10 | | 10 |

| | | | | |
|---|---|---|---|---|
| Total | 100 | 100 | 100 | 100 |
| Calculated ideal solubility* (% w/w) | 1.07 | 2.89 | 1.92 | 3.74 |
| Observed saturated solubility (% w/w) | 2.62 | 4.43 | 2.42 | 2.62 |
| Solubility enhancement ratio** | 2.5 | 1.5 | 1.3 | 0.7 |

*Calculated solubility = Σ(solubility of component * % w/w of component)
**Observed solubility/Calculated solubility
†Salicylic acid is a solid at room temperature, which prevents determination of solubility of imiquimod at room temperature in this component

As shown in Table 16, solubility enhancement was observed with 3 of the 4 formulations of the invention tested. The data of Table 16 indicates that the use of multiple hydrogen bond formers may generate additional solubility enhancement of imiquimod relative to corresponding individual hydrogen bond formers. The data suggests a synergistic enhancement in solubility of imiquimod when two hydrogen bond forming agents, such as oleth-3 phosphate and salicylic acid, are combined.

### EXAMPLE 9

### Imiquimod Enhanced Skin Penetration

A study was performed to characterize the in vitro percutaneous penetration of (¹⁴C)-Imiquimod from nine formulations of the invention compared to that from a commercial imiquimod product, Aldara® Cream, following topical application to excised human skin. The compositions of the formulations tested are shown in Table 17. Formulations of the invention contained 0.5 to 1% w/w imiquimod while Aldara® Cream contains 5% w/w imiquimod.

TABLE 17

| | | Formulation ID 2592- | | | | | | | | |
|---|---|---|---|---|---|---|---|---|---|---|
| | | 152A | 152B | 152C | 152D | 153A | 153B | 153C | 153D | 153E |
| Components | | % w/w | | | | | | | | |
| Imiquimod | Aldara | 1.0 | 1.0 | 0.5 | 0.5 | 0.5 | 0.5 | 1.0 | 0.5 | 1.0 |
| N-Methyl pyrrolidone (NMP) | | 95.0 | | | | 97.3 | 96.9 | 93.4 | 91.6 | 93.4 |
| Benzyl alcohol | | | 95.0 | | | | | | | |
| Transcutol | | | | 95.5 | | | | | | |
| Dimethyl isosorbide (DMI) | | | | | 95.5 | | | | | |
| Salicylic acid | | 4.0 | 4.0 | 4.0 | 4.0 | | | | | |
| Glycolic acid | | | | | | 2.2 | | | | |
| Lactic acid | | | | | | | 2.6 | | | |
| Glucuronic acid | | | | | | | | 5.6 | | |
| Laroyl sarcosine | | | | | | | | | 7.9 | |
| Oleth-3 phosphate | | | | | | | | | | 5.6 |
| Total | | 100.0 | 100.0 | 100.0 | 100.0 | 100.0 | 100.0 | 100.0 | 100.0 | 100.0 |

Dermatomed human skin was obtained from a single donor following elective abdominoplasty. The tissue was dosed with 5 mg/cm2 of formulation spiked with radiolabeled imiquimod at a nominal 1.0 μCi/dose, corresponding to a nominal 3.2 mg dose per cell. Percutaneous absorption was evaluated by mounting the dermatomed tissue in Bronaugh flow-through diffusion cells at 32° C. Five replicates were performed for each formulation. Fresh receptor fluid, PBS containing 0.1% w/v sodium azide and 1.5% w/v oleth-20, was continuously pumped under the skin at a nominal flow rate of 1 ml/hr and collected in 6-hour intervals. Following a 24-hour exposure period, the residual formulation remaining on the skin surface was removed by repeated tape stripping (3 strips/cell). Subsequently, the epidermis was physically separated from the dermis by gentle peeling. The quantity of radioactivity in the tape-strips, epidermis, dermis, and receptor phase samples was determined using liquid scintillation analyzing techniques. Dose recovery (accountability) at the end of the study ranged from 82.7 to 88.9 percent of the applied dose.

TABLE 18

| | | | Formulation ID 2592- | | | | | | | | |
|---|---|---|---|---|---|---|---|---|---|---|---|
| | | Aldara | 152A | 152B | 152C | 152D | 153A | 153B | 153C | 153D | 153E |
| Amount | % Applied | 0.238 | 4.56 | 0.757 | 0.5 | 0.198 | 17.9 | 20.2 | 3.77 | 4.99 | 11.3 |
| penetrated | dose | | | | | | | | | | |
| | ng/cm² | 594 | 2282 | 379 | 125 | 49.59 | 4485 | 5043 | 1883 | 1248 | 5638 |

As shown in Table 18, receptor phase levels of (¹⁴C)-Imiquimod from Aldara® Cream i.e. material that penetrated the skin was 0.238 (±0.04) percent of the applied dose (594+89 ng/cm2). Tissue permeation from the nine formulations of the invention containing 0.5 or 1% imiquimod ranged from 0.198 to 20.2 percent of the applied dose. The mass of imiquimod that penetrated the skin ranged from 49.6 to 5638 ng/cm². Thus, not only did a higher percentage of the applied dose of imiquimod penetrate into skin from 6 of the 9 formulations tested, but the absolute amount of imiquimod that penetrated into skin from 6 of the 9 formulations of the invention was about equal to or higher that that which penetrated into skin from the Aldara® Cream, even though the amount of imiquimod in the formulations of the invention contained only 10% or 20% of that contained in the prior art Aldara® Cream formulation.

### EXAMPLE 10

### Enhanced Solubility of Imiquimod

Several solvent mixtures of the invention were prepared to assess the solubilizing capacity of polar solvent compositions essentially free of water containing various hydrogen bond forming compounds. Solutions containing a blend of a multiplicity of non-aqueous solvents and a range of hydrogen bond forming compounds were prepared with excess imiquimod and incubated for approximately 1 week at 25° C., under constant agitation. Excess imiquimod was removed by centrifugation or filtration and the concentration of imiquimod in the clear supernatant was determined by HPLC-UV. The composition of each solution and the solubility of imiquimod in each solution are shown in table 19.

TABLE 19

| | Saturated | | | | | | | | | | | | | |
|---|---|---|---|---|---|---|---|---|---|---|---|---|---|---|
| | solubility | Formula ID 2737- | | | | | | | | | | | | |
| | (% w/w, | 59A | 59B | 59C | 60A | 60B | 60C | 61A | 61B | 61C | 62A | 62B | 62C | 63A |
| Component | 25° C.) | % w/w | | | | | | | | | | | | |
| NMP | 0.1683 | 30 | 30 | 30 | 30 | 30 | 30 | 30 | 50 | 30 | 30 | 30 | 30 | 30 |
| Benzyl | 0.4427 | 25 | 25 | 25 | 25 | 25 | 25 | 25 | 10 | 25 | 25 | 25 | 25 | 25 |
| Alcohol | | | | | | | | | | | | | | |
| Ethanol | 0.0294 | | | | | 36 | 32.5 | 25 | 23.5 | 18.5 | 13.5 | 32.5 | 32.5 | 28.5 |
| Glycerin | 0.1185 | 32.5 | 27.5 | 32.5 | 32.5 | | | | 5 | 10 | 10 | | | |
| Oleyl | 0.02065 | 5 | 10 | | | 5 | 5 | 5 | | 5 | 10 | | | |
| Alcohol^{a} | | | | | | | | | | | | | | |
| Oleth-10^{a} | 0.02065 | | | | 4 | | | | | | | | | |
| Oleth-2 | 0.02065 | | | 5 | 1 | | | | | | | | | |
| (BRIJ 93) | | | | | | | | | | | | | | |
| Oleth-3 | 5.6554 | | | | | | | | | | | 5 | 5 | 5 |
| Phosphate | | | | | | | | | | | | | | |
| N-Lauroyl | 11.4493 | 7.5 | 7.5 | 7.5 | 7.5 | | | | 7.5 | 7.5 | 7.5 | 7.5 | | 7.5 |
| Sarcosine^{b} | | | | | | | | | | | | | | |
| Salicylic | n/a‡ | | | | | 4 | 7.5 | 15 | 4 | 4 | 4 | | 7.5 | 4 |
| Acid | | | | | | | | | | | | | | |
| | Total | 100 | 100 | 100 | 100 | 100 | 100 | 100 | 100 | 100 | 100 | 100 | 100 | 100 |

| | | | | | | | | | | | | | |
|---|---|---|---|---|---|---|---|---|---|---|---|---|---|
| Calculated solubility* | 1.06 | 1.05 | 1.06 | 1.06 | 0.17 | 0.17 | 0.17 | 1.00 | 1.04 | 1.04 | 1.31 | 0.45 | 1.31 |
| (% w/w) | | | | | | | | | | | | | |
| Saturated Solubility | 2.53 | 2.63 | 2.71 | 2.65 | 1.08 | 1.13 | 1.29 | 1.29 | 2.03 | 1.95 | 3.62 | 2.50 | 3.25 |
| (% w/w) | | | | | | | | | | | | | |
| Solubility | 2.4 | 2.5 | 2.6 | 2.5 | 6.2 | 6.6 | 7.6 | 1.3 | 2.0 | 1.9 | 2.8 | 5.5 | 2.5 |
| enhancement | | | | | | | | | | | | | |
| ratio** | | | | | | | | | | | | | |

† - Salicylic acid is a solid at room temperature, which prevents determination of solubility of imiquimod at room temperature in this component

The data of Table 19 shows that an enhancement above calculated ideal solubility was obtained with all formulations of the invention.

### EXAMPLE 11

### Enhanced Delivery of Imiquimod from Formulations of the Invention

A study was performed to characterize the in vitro percutaneous penetration of (¹⁴C)-Imiquimod from nine formulations of the invention compared to that from a commercial imiquimod product, Aldara® Cream, following topical application to excised human skin. The compositions of the formulations tested and penetration data are shown in Table 20. Formulations of the invention contained 0.5 to 1.0% w/w imiquimod while Aldara® Cream contains 5% w/w imiquimod.

Dermatomed human skin was obtained from a single donor following elective abdominoplasty. The tissue was dosed with 5 mg/cm2 of formulation spiked with radiolabeled imiquimod at a nominal 1.0 μCi/dose, corresponding to a nominal 3.2 mg dose per cell. Percutaneous absorption was evaluated by mounting the dermatomed tissue in Bronaugh flow-through diffusion cells at 32° C. Five replicates were performed for each formulation. Fresh receptor fluid, PBS containing 0.1% w/v sodium azide and 1.5% w/v oleth-20, was continuously pumped under the skin at a nominal flow rate of 1 ml/hr and collected in 6-hour intervals. Following a 24-hour exposure period, the residual formulation remaining on the skin surface was removed by repeated tape stripping (3 strips/cell). Subsequently, the epidermis was physically separated from the dermis by gentle peeling. The quantity of radioactivity in the tape-strips, epidermis, dermis, and receptor phase samples was determined using liquid scintillation analyzing techniques. Dose recovery (accountability) at the end of the study ranged from 83.2 to 98.6 percent.

TABLE 20

| | | Formula ID 2737- | | | | | | | | |
|---|---|---|---|---|---|---|---|---|---|---|
| | | 74A | 74B | 74C | 75B | 76A | 77A | 87A | 77C | 78A |
| Component | % w/w | | | | | | | | | |
| Imiquimod | 5.0 | 2.0 | 2.0 | 2.0 | 1.0 | 1.0 | 1.75 | 2.75 | 2.0 | 2.5 |
| NMP | ALDARA | 30 | 30 | 30 | 30 | 30 | 30 | | 30 | 30 |
| Benzyl Alcohol | Lot: | 25 | 25 | 25 | 25 | 25 | 25 | 25 | 25 | 25 |
| Ethanol | IC107A, | | | | 34.5 | 23.5 | 11.25 | | 30 | 25.5 |
| Glycerin | Exp: | 30 | 25 | 30 | | | 10 | 39.2 | | |
| Transcutol | April | | | | | | | 25 | | |
| Oleyl Alcohol | 2009 | 5 | 10 | | 5 | 5 | 10 | | | |
| Oleth-2 (BRIJ 93) | | | | 5 | | | | | | |
| Oleth-3 Phosphate | | | | | | | | | 5 | 5 |
| N-Lauroyl Sarcosine | | 7.5 | 7.5 | 7.5 | | | 7.5 | 7.5 | | 7.5 |
| Salicylic Acid | | | | | 4 | 15 | 4 | | 7.5 | 4 |
| HPC | | 0.5 | 0.5 | 0.5 | 0.5 | 0.5 | 0.5 | 0.5 | 0.5 | 0.5 |
| | Total | 100.0 | 100.0 | 100.0 | 100.0 | 100.0 | 100.0 | 100.0 | 100.0 | 100.0 |
| Mean % applied | 0.045 | 0.384 | 0.424 | 0.295 | 0.081 | 0.159 | 0.563 | 0.084 | 0.023 | 0.069 |
| dose penetrated | | | | | | | | | | |
| Mean amount | 113 | 384 | 424 | 295 | 40 | 79 | 493 | 116 | 23 | 86 |
| penetrated, (ng/cm²) | | | | | | | | | | |

As shown in Table 20, receptor phase levels of (¹⁴C)-Imiquimod from Aldara® Cream, was 0.0536 percent of the applied dose which corresponded to 134 ng/cm². Tissue permeation of (¹⁴C)-Imiquimod ranged from 0.023 to 0.563 percent of the applied dose (equivalent to 23.3 ng/cm² and 493 ng/cm² of Imiquimod) from formulations of the invention. Formulations 2737-74B and 2737-77A generated the highest permeation amount of (¹⁴C)-Imiquimod with 0.424 and 0.563 percent of the applied dose (equivalent to 424 ng/cm² and 493 ng/cm² of Imiquimod), respectively. Thus, not only did a higher percentage of the applied dose of imiquimod penetrate into skin from 8 of the 10 formulations tested but the absolute amount of imiquimod that penetrated into skin from 5 of the 10 formulations of the invention was about equal to or higher that that which penetrated into skin from the Aldara® Cream, even though the amount of imiquimod in the formulations of the invention contained only 10% or 20% of that contained in the Aldara® Cream formulation.

### EXAMPLE 12

### Enhanced Stability of Imiquimod Formulations of the Invention

Based on skin penetration and preliminary physical stability data, two formulations of the invention, 2737-77A and 2737-74B, were evaluated for stability characteristics. Samples were placed at freeze/thaw cycling (F/T), 5° C., 25° C., 40° C. and 50° C. and were observed and assayed at periods up to 12 weeks. The stability data for formulation 2737-77A and 2737-74B is summarized in Tables 21 and 22, respectively.

TABLE 21

| | | | | Assay |
|---|---|---|---|---|
| Batch | | | | (% label |
| No. | Condition | Pull Point | Observations | claim) |
| 2737-77A | T = 0 | Initial | Clear colorless solution | 99.94 |
| | F/T | 4 weeks | Colorless w/white ppt | N/T |
| | F/T | 4 cycles | No adverse observations | N/T |
| | 5° C. | 4 weeks | N/T | N/T |
| | | 8 weeks | Light yellow, with ppt | 59.26 |
| | | 12 weeks | Light yellow, with ppt | 95.71 |
| | 25° C. | 4 weeks | Clear light yellow solution | 100.07 |
| | | 8 weeks | Clear light yellow solution | 100.14 |
| | | 12 weeks | Clear light yellow solution | N/T |
| | 30° C. | 4 weeks | N/A | N/T |
| | | 8 weeks | Clear light yellow solution | N/T |
| | | 12 weeks | Clear light yellow solution | 97.99 |
| | 40° C. | 4 weeks | Clear light yellow solution | 99.83 |
| | | 8 weeks | Clear light yellow solution | 99.81 |
| | | 12 weeks | Clear light yellow solution | 97.7 |
| | 50° C. | 4 weeks | Clear light yellow solution | 99.44 |
| | | 8 weeks | Clear light yellow solution | 99.23 |
| | | 12 weeks | Clear yellow solution | 96.85 |

N/A—not applicable;
N/T not tested

TABLE 22

| | | | | Assay |
|---|---|---|---|---|
| | | | | Imiquimod |
| Batch | | | | (% label |
| No. | Condition | Pull Point | Observations | claim) |
| 2737- | T = 0 | Initial | Clear colorless solution | 98.80 |
| 74B | F/T | 4 weeks | Clear colorless solution | N/T |
| | F/T | 4 cycles | N/T | N/T |
| | 5° C. | 4 weeks | N/T | 99.35 |
| | | 8 weeks | Clear light yellow solution | 99.31 |
| | | 12 weeks | Clear light yellow solution | 97.87 |
| | 25° C. | 4 weeks | Clear light yellow solution | 99.03 |
| | | 8 weeks | Clear light yellow solution | 99.36 |
| | | 12 weeks | Clear light yellow solution | 99.68 |
| | 30° C. | 4 weeks | N/A | N/T |
| | | 8 weeks | Clear light yellow solution | N/T |
| | | 12 weeks | Clear light yellow solution | 97.85 |
| | 40° C. | 4 weeks | Clear light yellow solution | 98.53 |
| | | 8 weeks | Clear light yellow solution | 98.58 |
| | | 12 weeks | Clear light yellow solution | 96.76 |
| | 50° C. | 4 weeks | Clear light yellow solution | 97.43 |
| | | 8 weeks | Clear yellow solution | 97.71 |
| | | 12 weeks | Clear yellow solution | 96.17 |

N/A—not applicable;
N/T not tested

As shown in Tables 21 and 22, the formulations of the invention exhibited good physical and chemical stability for a period of 12 weeks at all temperatures tested. The stability testing at accelerated conditions at 40° C. and 50° C. indicate that the formulations of the invention are stable at lower temperatures, such as at room temperature, for durations much longer than 12 weeks. The presence of a polyol such as glycerin appears to enhance the physical stability of formulations of the invention with respect to precipitation of imiquimod over time.

### EXAMPLE 13

### Enhanced Solubility and Stability of Formulations of the Invention with Increased Concentration of Volatile Solvent

The addition of increasing amounts of volatile components to formulations is often useful as it can improve skin application parameters such as ease of rub-in. Volatile components such as an alcohol such as ethanol volatilize rapidly following unoccluded topical application and reduce the amount of residual non-volatile material that requires rub-in and absorption into the skin. Enhanced ease of application can improve patient compliance and thus efficacy.

In order to evaluate the amount of volatile components, such as ethanol, that can be included in formulations of the invention, a representative base formulation was selected and modified. The base formulation composition is listed in Table 23 and demonstrated synergistic solubility enhancement, required physical stability (no precipitation at 25° C. and 40° C. for 12 weeks), and highly efficient skin penetration (greater efficiency than Aldara® cream).

TABLE 23

| Component | % w/w |
|---|---|
| Imiquimod | 1.75 |
| NMP | 30 |
| Benzyl Alcohol | 25 |
| Ethanol | 11.25 |
| Glycerin | 10 |
| Oleyl Alcohol | 10 |
| N-Lauroyl Sarcosine | 7.5 |
| Salicylic Acid | 4 |
| HPC | 0.5 |
| Total | 100.0 |

The compositions of the formulas that were tested are listed in Table 24 and contained 2.2 to 3.0% w/w Imiquimod and 34 to 60% w/w ethanol as the volatile component. It was determined that it was possible to dissolve 1.3 to 1.7 times more imiquimod in the modified compositions than in the base formulation, thereby demonstrating synergistic solubility enhancement due to the presence of increased concentrations of the volatile component. The formulae were also physically stable as they did not exhibit precipitation after 12 weeks at 5° C., 25° C., 40° C. and 50° C.

TABLE 24

| | Formulation ID 2828- | | | | | | | | | |
|---|---|---|---|---|---|---|---|---|---|---|
| | 1A | 1B | 1C | 1D | 2A | 2B | 2C | 2D | 3A | 3B |
| Component | % w/w | | | | | | | | | |
| Imiquimod | 2.75 | 2.75 | 3.0 | 2.5 | 2.75 | 2.75 | 2.5 | 2.5 | 2.5 | 2.5 |
| NMP | 14.1 | 11.6 | 9.1 | 19.1 | 14.1 | 9.1 | 14.1 | 14.1 | 11.6 | 11.6 |
| Benzyl alcohol | 9.75 | 9.75 | 9.5 | | 9.75 | 9.75 | 5 | | 7.5 | 2.5 |
| Ethanol | 50 | 50 | 50 | 50 | 55 | 55 | 55 | 55 | 60 | 60 |
| Glycerin | 10 | 10 | 10 | 10 | 10 | 10 | 10 | 10 | 10 | 10 |
| N-Lauroyl sarcosine | 7.5 | 7.5 | 7.5 | 7.5 | 7.5 | 7.5 | 7.5 | 7.5 | 7.5 | 7.5 |
| Salicylic acid | 0.4 | 0.4 | 0.4 | 0.4 | 0.4 | 0.4 | 0.4 | 0.4 | 0.4 | 0.4 |
| Oleyl alcohol | 5 | 7.5 | 10 | 10 | 0 | 5 | 5 | 10 | 0 | 5 |
| HPC | 0.5 | 0.5 | 0.5 | 0.5 | 0.5 | 0.5 | 0.5 | 0.5 | 0.5 | 0.5 |
| Total | 100 | 100 | 100 | 100 | 100 | 100 | 100 | 100 | 100 | 100 |

| | Formulation ID 2828- | | | | | | | | |
|---|---|---|---|---|---|---|---|---|---|
| | 3C | 3D | 7A | 7B | 7C | 8C | 9A | 9C | 10A |
| Component | % w/w | | | | | | | | |
| Imiquimod | 2.5 | 2.5 | 2.5 | 2.5 | 2.2 | 2.5 | 2.5 | 2.9 | 2.9 |
| NMP | 14.1 | 9.1 | 20 | 20 | 20 | 25 | 25 | 15 | 15 |
| Benzyl alcohol | 5 | 5 | | | | 5 | 5 | 9.6 | 9.2 |
| Ethanol | 60 | 60 | 47 | 46.6 | 46.9 | 34.5 | 34.1 | 39.5 | 39.5 |
| Glycerin | 10 | 10 | 12.5 | 12.5 | 12.5 | 15 | 15 | 15 | 15 |
| N-Lauroyl sarcosine | 7.5 | 7.5 | 7.5 | 7.5 | 7.5 | 7.5 | 7.5 | 7.5 | 7.5 |
| Salicylic acid | 0.4 | 0.4 | | 0.4 | 0.4 | | 0.4 | | 0.4 |
| Oleyl alcohol | 0 | 5 | 10 | 10 | 10 | 10 | 10 | 10 | 10 |
| HPC | 0.5 | 0.5 | 0.5 | 0.5 | 0.5 | 0.5 | 0.5 | 0.5 | 0.5 |
| Total | 100 | 100 | 100 | 100 | 100 | 100 | 100 | 100 | 100 |

### EXAMPLE 14

### Comparable Skin Delivery of Imiquimod from Formulations of the Invention Containing more than 30% Volatile Components

A study was performed to characterize the in vitro percutaneous penetration of (¹⁴C)-Imiquimod from eight formulations of the invention containing increased concentrations of volatile components compared to the penetration from a commercial imiquimod product, Aldara® Cream, following topical application to excised human skin. Several formulations from Example 13 were selected for skin penetration evaluation using compositional variation and physical stability as selection criteria. The compositions of the formulations tested and penetration data are shown in Table 25. Formulations of the invention contained 2.5 to 3.0% w/w imiquimod while Aldara® Cream contained 5% w/w imiquimod.

Dermatomed human skin was obtained from a single donor following elective abdominoplasty. The tissue was dosed with 5 mg/cm² of formulation spiked with radiolabeled imiquimod at a nominal 1.0 μCi/dose, corresponding to a nominal 3.2 mg dose per cell. Percutaneous absorption was evaluated by mounting the dermatomed tissue in Bronaugh flow-through diffusion cells at 32° C. Six replicates were performed for each formulation. Fresh receptor fluid, PBS containing 0.1% w/v sodium azide and 1.5% w/v oleth-20, was continuously pumped under the skin at a nominal flow rate of 1 ml/hr and collected in 6-hour intervals. Following a 24-hour exposure period, the residual formulation remaining on the skin surface was removed by repeated tape stripping (3 strips/cell). Subsequently, the epidermis was physically separated from the dermis by gentle peeling. The quantity of radioactivity in the tape-strips, epidermis, dermis, and receptor phase samples was determined using liquid scintillation analyzing techniques. Dose recovery (accountability) at the end of the study ranged from 73.6 to 83.5 percent of the applied dose.

TABLE 25

| | | Formulation ID | | | | | | | |
|---|---|---|---|---|---|---|---|---|---|
| | | 1B | 1C | 2A | 2B | 3A | 3C | 9A | 10A |
| Component | Control | % w/w | | | | | | | |
| Imiquimod | Aldara | 2.75 | 3.0 | 2.75 | 2.75 | 2.5 | 2.5 | 2.5 | 2.9 |
| NMP | Lot #: | 11.6 | 9.1 | 14.1 | 9.1 | 11.6 | 14.1 | 25.0 | 15.0 |
| Benzyl alcohol | ID107A; | 9.75 | 9.5 | 9.75 | 9.75 | 7.5 | 5 | 5.0 | 9.2 |
| Ethanol | Exp: | 50 | 50 | 55 | 55 | 60 | 60 | 34.1 | 39.5 |
| Glycerin | April | 10 | 10 | 10 | 10 | 10 | 10 | 15 | 15 |
| N-Lauroyl sarcosine | 2009 | 7.5 | 7.5 | 7.5 | 7.5 | 7.5 | 7.5 | 7.5 | 7.5 |
| Salicylic acid | | 0.4 | 0.4 | 0.4 | 0.4 | 0.4 | 0.4 | 0.4 | 0.4 |
| Oleyl alcohol | | 7.5 | 10 | 0 | 5 | 0 | 0 | 10.0 | 10.0 |
| HPC (HXF grade) | | 0.5 | 0.5 | 0.5 | 0.5 | 0.5 | 0.5 | 0.5 | 0.5 |
| Total | | 100.0 | 100.0 | 100.0 | 100.0 | 100.0 | 100.0 | 100.0 | 100. |
| Amount Penetrated | 0.168 | 0.181 | 0.213 | 0.113 | 0.187 | 0.137 | 0.105 | 0.223 | 0.21 |
| (% applied dose) | | | | | | | | | |
| Amount Penetrated | 420 | 248 | 319 | 156 | 257 | 172 | 131 | 279 | 305 |
| (ng/cm²) | | | | | | | | | |

As shown in Table 25, receptor phase levels of (¹⁴C)-Imiquimod from Aldara® Cream was 0.168 percent of the applied dose which corresponded to 420 ng/cm² of imiquimod. Tissue permeation of (¹⁴C)-Imiquimod ranged from 0.105 to 0.223 percent of the applied dose (equivalent to 131 ng/cm to 279 ng/cm² of imiquimod) from formulations of the invention. Formulations 2828-IC (3% Imiquimod) and 2828-9A (2.5% Imiquimod) had the highest efficiency in permeation of (¹⁴C)-Imiquimod with 0.213 and 0.223 percent of the applied dose (equivalent to 319 ng/cm² and 279 ng/cm² of Imiquimod), respectively. Delivery efficiency (percent applied dose) from 5 of the 8 formulae of the invention was greater than from the Aldara® Cream.

### EXAMPLE 15

### Formulations of the Invention with Low Levels of Fatty Acids that are Liquid at Room Temperature

The following formulations of the invention as shown in Table 26 are made containing low levels of one or more fatty acids that are liquid at room temperature. The formulations provide enhanced solubility of imiquimod and good physical stability.

TABLE 26

| | | | |
|---|---|---|---|
| #1 | #2 | #3 | #4 |

| | |
|---|---|
| Component | % w/w |

| | | | | |
|---|---|---|---|---|
| Imiquimod | 1.5 | 1.5 | 1.5 | 1.5 |
| NMP | 30 | 30 | 30 | 30 |
| Benzyl Alcohol | 25 | 25 | 25 | 25 |
| Ethanol | 26 | 21 | 26 | 25.5 |
| Oleyl Alcohol | 5 | 10 | 5 | 5 |
| N-Lauroyl | | 7.5 | 5 | 5 |
| Sarcosine | | | | |
| Isostearic acid | 10 | 5 | 5 | 5 |
| Oleic acid | 2.5 | | 2.5 | 2.5 |
| HPC | | | | 0.5 |
| Total | 100.00 | 100.00 | 100.00 | 100.00 |

Further modifications, uses, and applications of the invention described herein will be apparent to those skilled in the art. It is intended that such modifications be encompassed in the above description and in the following claims.

1. A solution comprising one or more polar solvents other than water, imiquimod or an analog thereof dissolved within the one or more solvents, and a hydrogen bond forming compound dissolved in the one or more solvents, and optionally water wherein the solution contains 30% w/w or less water.

2. The solution of claim 1 which is essentially free of water.

3. The solution of claim 1 which contains water.

4. The solution of claim 3 wherein the concentration of water contained therein is less than 5 times the concentration of the imiquimod or analog thereof contained in the solution.

5. The solution of claim 3 wherein the solution contains less than 25% water.

6. The solution of claim 5 wherein the solution contains less than 20% water.

7. The solution of claim 6 wherein the solution contains less than 15% water.

8. The solution of claim 1 which contains a low level of fatty acids that are liquid at room temperature.

9. The solution of claim 8 which is substantially free of fatty acids that are liquid at room temperature.

10. The solution of claim 1 wherein the molar ratio of the imiquimod or analog thereof and the hydrogen bond forming compound is between 4:1 and 1:4.

11. The solution of claim 1 wherein the hydrogen bond forming compound is selected from the group consisting of alpha-hydroxy acids, beta-hydroxy acids, alkyl-sarcosinates, anionic pegylated dimethicone derivatives, anionic oleyl ether surfactants, anionic laureth ether surfactants, cyclic acids, and cyclic acidic sugars.

12. The solution of claim 1 wherein the solvent is selected from the group consisting of aprotic solvents, cyclic alcohols, short chain liquid alcohols, diols, triols, esters, ethers, pharmaceutical oils, and silicones.

13. The solution of claim 1 which comprises a polymer having a solubility of at least 0.01% in the one or more solvents.

14. The solution of claim 13 wherein the polymer is selected from the group consisting of cellulose derivatives, methacrylic acid copolymers, carbomers, pyrrolidone-containing polymers, polyoxyethylene/polyoxypropylene block co-polymers, and polyvinyl alcohols.

15. The solution of claim 13 wherein the concentration of the polymer in the solution is less than 10% w/w.

16. The solution of claim 1 which comprises a multiplicity of solvents.

17. The solution of claim 1 which forms an external or internal phase of an emulsion.

18. The solution of claim 16 which comprises at least 30% w/w of a volatile solvent.

19. The solution of claim 20 wherein the volatile solvent is alcohol.

20. The solution of claim 1 which comprises a polyol.

21. The solution of claim 20 wherein the polyol is glycerin.

22. A method for making a solution comprising combining one or more polar solvents other than water and dissolving in the one or more solvents imiquimod or an analog thereof and a hydrogen bond forming compound in an amount sufficient to increase the solubility of the imiquimod or analog in the solution, and optionally combining water in the solution at a concentration of 30% or less w/w of the solution.

23. The method of claim 22 wherein essentially no water is combined in the solution.

24. The method of claim 22 wherein water is combined in the solution.

25. The method of claim 24 wherein the concentration of water that is combined in the solution is less than 5 times the concentration of the imiquimod or analog thereof combined in the solution.

26. The method of claim 24 wherein water is combined at a concentration of less than 25% w/w of the solution.

27. The method of claim 26 wherein water is combined at a concentration of less than 20% w/w of the solution.

28. The method of claim 27 wherein water is combined at a concentration of less than 15% w/w of the solution.

29. The method of claim 22 wherein fatty acids that are liquid at room temperature are combined in the solution at a concentration of 12.5% w/w or less.

30. The method of claim 22 wherein substantially no fatty acids that are liquid at room temperature are combined in the solution.

31. The method of claim 22 wherein the imiquimod or analog thereof and the hydrogen bond forming compound are combined in a molar ration between 4:1 and 1:4.

32. The method of claim 22 wherein the hydrogen bond forming compound is selected from the group consisting of alpha-hydroxy acids, beta-hydroxy acids, alkyl-sarcosinates, anionic pegylated dimethicone derivatives, anionic oleyl ether surfactants, anionic laureth ether surfactants, cyclic acids, and cyclic acidic sugars.

33. The method of claim 22 wherein the non-aqueous solvent is selected from the group consisting of aprotic solvents, cyclic alcohols, short chain liquid alcohols, diols, triols, esters, ethers, pharmaceutical oils, and silicones.

34. The method of claim 22 wherein a polymer having a solubility of at least 0.0 I% is combined in the solution.

35. The method of claim 34 wherein the polymer is selected from the group consisting of cellulose derivatives, methacrylic acid copolymers, carbomers, pyrrolidone-containing polymers, polyoxyethylene/polyoxypropylene block co-polymers, and polyvinyl alcohols.

36. The method of claim 34 wherein the concentration of the polymer in the solution is less than 10% w/w.

37. The method of claim 22 wherein a multiplicity of polar solvents other than water are combined.

38. The method of claim 37 wherein at least one of the polar solvents other than water is a volatile solvent at a concentration higher than 30%.

39. The method of claim 38 wherein the volatile solvent is an alcohol.

40. The method of claim 22 wherein a polyol is combined in the solution.

41. The method of claim 40 wherein the polyol is glycerin.

42. A method for increasing the skin penetration of imiquimod or an analog thereof comprising topically administering a pharmaceutical formulation comprising a solution comprising one or more polar solvents other than water, imiquimod or an analog thereof dissolved within the one or more solvents, and a hydrogen bond forming compound dissolved in the one or more solvents, and optionally water wherein the solution contains 30% w/w or less water.

43. The method of claim 42 wherein the solvent is selected from the group consisting of aprotic solvents, cyclic alcohols, short chain liquid alcohols, diols, triols, esters, ethers, pharmaceutical oils, and silicones.

44. The method of claim 42 wherein the solution is essentially free of water.

45. The method of claim 42 wherein the solution contains water.

46. The method of claim 45 wherein the concentration of water in the solution is less than 5 times the concentration of the imiquimod or analog thereof contained in the solution.

47. The method of claim 45 wherein the concentration of water in the solution is less than 25%.

48. The method of claim 47 wherein the concentration of water in the solution is less than 20%.

49. The method of claim 48 wherein the concentration of water in the solution is less than 15%.

50. The method of claim 42 wherein the solution contains a low level of fatty acids that are liquid at room temperature.

51. The method of claim 42 wherein the solution is substantially free of fatty acids that are liquid at room temperature.

52. The method of claim 42 wherein the molar ratio of the imiquimod or analog thereof and the hydrogen bond forming compound in the solution is between 4:1 and 1:4.

53. The method of claim 42 wherein the hydrogen bond forming compound is selected from the group consisting of alpha-hydroxy acids, beta-hydroxy acids, alkyl-sarcosinates, anionic pegylated dimethicone derivatives, anionic oleyl ether surfactants, anionic laureth ether surfactants, cyclic acids, and cyclic acidic sugars.

54. The method of claim 42 wherein the solvent is selected from the group consisting of aprotic solvents, cyclic alcohols, short chain liquid alcohols, diols, triols, esters, ethers, pharmaceutical oils, and silicones.

55. The method of claim 42 wherein the solution comprises a polymer having a solubility of at least 0.01% in the one or more solvents.

56. The method of claim 55 wherein the polymer is selected from the group consisting of cellulose derivatives, methacrylic acid copolymers, carbomers, pyrrolidone-containing polymers, polyoxyethylene/polyoxypropylene block co-polymers, and polyvinyl alcohols.

57. The method of claim 55 wherein the concentration of the polymer in the solution is less than 10% w/w.

58. The method of claim 42 wherein the solution comprises a multiplicity of solvents other than water.

59. The method of claim 42 wherein the solution forms an external or internal phase of an emulsion.

60. The method of claim 58 wherein at least one of the solvents is a volatile solvent at a concentration in the solution of at least 30% w/w.

61. The method of claim 60 wherein the volatile solvent is an alcohol.

62. The method of claim 42 wherein the solution comprises a polyol.

63. The method of claim 62 wherein the polyol is glycerin.
